(19) United States
(12) Patent Application Publication
(10) Pub. No.: US 2006/0177502 A1
(43) Pub. Date: Aug. 10, 2006

(54) Sustained release pharmaceutical formulations

(76) Inventors: Srikonda Sastry, Sunnyvale, CA (US); Janaki Nyshadham, Fremont, CA (US)

Correspondence Address: CV Therapeutics, Inc., 3172 Porter Drive, Palo Alto, CA, 94304, US

(21) Appl. No.: 11/326,965

(22) Filed: Jan. 5, 2006

Related U.S. Application Data
(60) Provisional application No. 60/642,168, filed on Jan. 6, 2005.

(51) Int. Cl. A61K 9/20

(52) U.S. Cl. 424/464

(57) ABSTRACT

Disclosed are novel ranolazine sustained release pharmaceutical formulations.

### BACKGROUND

Priority is claimed to U.S. Provisional Patent Application Ser. No. 60/642,168, filed Jan. 6, 2005, the complete disclosure of which is hereby incorporated by reference.

Ranolazine (N-(2,6-dimethylphenyl)-4-[2-hydroxy-3-(2-methoxyphenoxy)-propyl]-1-piperazineacetamide) is an agent that has been found to be useful for treating many disease states, including heart failure, arrhythmias, angina, diabetes, myocardial infarction, intermittent claudication, and the like. Ranolazine has been the subject of clinical trials for the treatment of some of these disease states, including angina, in particular chronic angina.

Initially, the clinical trial of ranolazine on humans suffering from angina was thought to be a failure, because the use of an immediate release ranolazine formulation at a dose level of 120 mg taken three times daily was ineffective. Later clinical work carried out with ranolazine clearly demonstrated that, as a consequence of the relatively short half life of ranolazine, for effective treatment of angina it is necessary to provide a delivery system that maintains satisfactory plasma levels of ranolazine over an extended period of time, i.e., via a sustained release formulation.

Ranolazine sustained release formulations have previously been disclosed—for example, see U.S. Pat. No. 5,506,229, in which a controlled release formulation in capsule form is disclosed, comprising microspheres of ranolazine and microcrystalline cellulose coated with release controlling polymers. In clinical trials such formulations were not successful in providing satisfactory plasma levels of ranolazine over an extended period of time. U.S. Pat. No. 6,503,911 disclosed sustained release formulations that overcame the problem of affording a satisfactory plasma level of ranolazine while the formulation travels through both an acidic environment in the stomach and a more basic environment through the intestine, and have proved to be very effective in providing the plasma levels that are necessary for the treatment of angina and other cardiovascular diseases. However, such formulations are composed of a relatively complex mixture of ingredients, and it would be desirable to provide a ranolazine sustained release formulation that utilizes fewer components and is simpler to manufacture, but nonetheless provides plasma levels that are effective for the treatment of angina and other cardiovascular diseases over long periods of time, particularly over a 24 hour period.

The sustained release ranolazine formulations of U.S. Pat. No. 6,503,911 were disclosed to comprise a mixture of ranolazine and a partially neutralized pH-dependent binder that controls the rate of ranolazine dissolution in aqueous media across the range of pH in the stomach (typically approximately 1-2) and in the intestine (typically approximately about 5.5). It was stated that the dosage forms of this invention require at least one pH-dependent binder, preferably in combination with a pH-independent binder, and that the ranolazine content of the formulations ranges from about 50% by weight to about 95% or more by weight, more preferably between about 70% to about 90% by weight and most preferably from about 70 to about 80% by weight.

Surprisingly, it has been discovered that ranolazine sustained release formulations can be prepared that provide the appropriate plasma levels of ranolazine that are necessary for the treatment of angina and other cardiovascular diseases, but do not require all of the components of the SR formulations disclosed in U.S. Pat. No. 6,503,911. In particular, it has been discovered that ranolazine SR formulations can be prepared that provide effective plasma levels of ranolazine for the treatment of angina and other cardiovascular diseases over long periods of time that do not require a pH dependent binder. It has also been discovered that effective ranolazine SR formulations can be prepared with a ranolazine content below 50%.

### SUMMARY OF THE INVENTION

In a first aspect, the invention relates to oral ranolazine sustained release formulations that provide therapeutic plasma levels of ranolazine for at least 12 hours when administered to a mammal, comprising formulations that contain less than 50% ranolazine, for example about 35-50%, preferably about 40-45% ranolazine.

In one embodiment the ranolazine sustained release formulations of the invention include a pH dependent binder; a pH independent binder; and one or more pharmaceutically acceptable excipients. Suitable pH dependent binders include, but are not limited to, a methacrylic acid copolymer, for example Eudragit® (Eudragit® L100-55, pseudolatex of Eudragit® L100-55, and the like) partially neutralized with a strong base, for example sodium hydroxide, potassium hydroxide, or ammonium hydroxide, in a quantity sufficient to neutralize the methacrylic acid copolymer to an extent of about 1-20%, for example about 306%. Suitable pH independent binders include, but are not limited to, hydroxypropylmethylcellulose (HPMC), for example Methocel® El OM Premium CR grade HPMC or Methocel® E4M Premium HPMC. Suitable pharmaceutically acceptable excipients include magnesium stearate and microcrystalline cellulose (Avicel®b pH101).

In a second aspect, the invention relates to oral ranolazine sustained release formulations that provide therapeutically effective plasma levels of ranolazine for at least 12 hours when administered, comprising formulations that contain at least about 35% ranolazine, preferably about 40-80% ranolazine, a pH independent binder, and one or more pharmaceutically acceptable excipients. Preferably the pH independent binder has a viscosity of about 4,000-12,000 cPs. Suitable pH independent binders include hydroxypropylmethylcellulose (HPMC), for example Methocel® E10M Premium CR grade HPMC or Methocel® E4M Premium HPMC. Examples of pharmaceutically acceptable excipients include magnesium stearate, microcrystalline cellulose, sodium alginate, xanthen, lactose, and the like.

In a third aspect, the invention relates to the use of the oral ranolazine sustained release formulations for the treatment of various disease states, especially cardiovascular diseases, for example heart failure, including congestive heart failure, acute heart failure, myocardial infarction, and the like, arrhythmias, angina, including exercise-induced angina, variant angina, stable angina, unstable angina, acute coronary syndrome, and the like, diabetes, and intermittent claudication. The treatment of such disease states is disclosed in various U.S. patents and patent applications, including U.S. Pat. No. 6,503,911, U.S. patent application Nos. 2003-0220344 and 2004-0063717, the complete disclosures of which are hereby incorporated by reference.

### Definitions and General Parameters

As used in the present specification, the following words and phrases are generally intended to have the meanings as set forth below, except to the extent that the context in which they are used indicates otherwise.

Examples of pH-dependent binder materials suitable for this invention include, but are not limited to, phthalic acid derivatives of vinyl polymers and copolymers, hydroxyalkylcelluloses, alkylcelluloses, cellulose acetates, hydroxyalkylcellulose acetates, cellulose ethers, alkylcellulose acetates, and the partial esters thereof, and polymers and copolymers of lower alkyl acrylic acids and lower alkyl acrylates, and the partial esters thereof.

Particularly suitable is methacrylic acid copolymer, type C, USP (Eudragit® L 100-55 or a pseudolatex of Eudragit® L100-55), which is a copolymer of methacrylic acid and ethyl acrylate having between 46.0% and 50.6% methacrylic acid units. Such a copolymer is commercially available, from Rohm Pharma as Eudragit® RTM. L 100-55 (as a powder) or L30D-55 (as a 30% dispersion in water). Other examples of pH-dependent binder materials that may be used alone or in combination in a sustained release ranolazine dosage form include, but are not limited to, hydroxypropyl cellulose phthalate, hydroxypropyl methylcellulose phthalate, cellulose acetate phthalate, polyvinylacetate phthalate, polyvinylpyrrolidone phthalate, and the like.

Examples of pH-independent binder materials suitable for this invention include but are not limited to, hydroxypropylmethylcellulose, hydroxyethylcellulose, hydroxypropylcellulose, methylcellulose, polyvinylpyrrolidone, neutral polymethacrylate esters, and the like. Particularly suitable is hydroxypropylmethylcellulose (HPMC), which is available from, for example, the Dow Chemical Company in various grades—for example, Methocel® E5 Premium LV JP, Methocel® E10M Premium CR grade, Methocel® E5 Premium LV EP JP, and the like. Those pH-independent binders that have a viscosity of about of about 4,000-12,000 cPs are preferred (viscosity as measured as a 2% solution of the binder in water at 20° C.). Examples of such pH independent binders include but are not limited to, hydroxypropylmethylcellulose (HPMC), for example Methocel® E10M Premium CR grade HPMC or Methocel® E4M Premium HPMC, which may be purchased from the Dow Chemical Company.

### EXAMPLE 1

Preparation of a 40% Ranolazine SR Formulation

Procedure:

Ranolazine (400 g), Avicel® (212.3 g), Eudragit® L100-55 (350 g), and hydroxypropylmethylcellulose HPMC (10.7 g) were mixed together in a granulator (KG-5 high shear mixer) for five minutes at 250 rpm impeller speed and 2500 rpm chopper speed.

Sodium hydroxide (6.67 g) was dissolved in 230 ml of water, and the solution was added to the powder mix at a rate of 50 ml/minute, impeller speed of 500 rpm and chopper speed of 10000 rpm.

A further amount of water (30 ml) was added at the rate of 100 ml/min, with an impeller speed of 500 rpm and chopper speed of 10000 rpm.

Powder was massed at impeller speed of 250 rpm and chopper speed 5000 rpm for 15 minutes in order to facilitate agglomeration.

The granules prepared in the previous step were dried in a fluid bed dryer for 25 minutes at an inlet air temperature of 60° C. and a nominal air flow setting of 8.

The dried granules were passed through a screen mill using an appropriate screen (0.083 inch screen).

The granules obtained were taken out, weighed and mixed with magnesium stearate (20 g, presifted with 40 mesh) for 3 minutes using a blender.

The granules weighing 1250 mg were compressed at a compression pressure ranging from 2500 to 3500 lb using a semiautomated Carver press to provide ranolazine SR tablets with 40% drug loading.

Preparation of other SR Formulation with Less than 50% Ranolazine

Similarily, other sustained release formulations with amounts of ranolazine ranging from 35%-50% are prepared.

Dissolution Test

Tablets were tested using United States Pharmacopeia dissolution apparatus type II (which is a standard apparatus for testing dissolution rates), stirring at 50 rpm. 900 ml of 0.1 N hydrochloric acid was the dissolution medium at a temperature of 37° C. 3 ml samples were taken at different intervals and replaced by fresh medium. Samples were analyzed at 272 nm.

Results:

| Time | 75% Ranolazine | 40% Ranolazine |
|---|---|---|
| (hr) | Drug Release (%) | Drug Release (%) |
| 0.5 | 17.2 | 29.4 |
| 1 | 25.4 | 35.9 |
| 2 | 36.0 | 44.8 |
| 4 | 49.0 | 56.0 |
| 6 | 57.0 | 64.8 |
| 8 | 63.0 | 72.0 |
| 12 | 73.0 | 80.4 |
| 20 | 87.0 | 89.2 |
| 24 | 93.0 | 89.2 |
| F1 (<15) | | 13.7 |
| F2 (>50) | | 54.2 |

Conclusion

The above table compares the dissolution profile of 40% SR ranolazine with 75% SR ranolazine, which is the formulation used in clinical testing of ranolazine. The F values are known as “fit factors”, as disclosed in Moore, J. W, and H. H. Flanner, 1996, “Mathematical Comparison of Dissolution Profiles”, Pharmaceutical Technology, 20 (6):64-74, the complete disclosure of which is hereby incorporated by reference. For the formulations to be considered comparable with respect to their dissolution profile, F1 should have a numerical value of less than 15 and F2 should have a numerical value of greater than 50. As can be seen from the above table, the 40% formulation and the 75% formulation are comparable using these criteria.

### EXAMPLE 2

Preparation of a Ranolazine SR Formulation with a pH Independent Polymer

Procedure:

Ranolazine (750 g) and hydroxypropylmethylcellulose (HPMC E10M Premium CR, 230 g) were mixed together in a granulator (KG-5 high shear mixer) for five minutes at 250 rpm impeller speed and 2500 rpm chopper speed.

260 ml of water was added to the powder mixture at a rate of 100 ml/min, impeller speed of 250 rpm, and chopper speed of 2500.

Powder was massed at impeller speed of 250 rpm and chopper speed 5000 rpm for 5 minutes rpm in order to facilitate agglomeration.

The granules prepared in Step 3 were dried in a fluid bed dryer for 25 minutes at an inlet air temperature of 60° C. and a nominal air flow setting of 8.

The dried granules were passed through a screen mill using an appropriate screen (0.083 inch screen).

The granules obtained were taken out, weighed and mixed with 2% magnesium stearate (20 g, presifted with 40 mesh) for 3 minutes using a blender (for example, a V-blender).

Granules weighing 666.7 mg were compressed on a Carver Press, then on a Stokes 16-Station press with 4 punch set.

Similarly, other Ranolazine SR Formulations are prepared with a pH Independent Polymer, varying the amounts of ranolazine and/or the pH independent binder.

Dissolution Test

Tablets were tested using USP dissolution apparatus II, stirring at 50 rpm. Using 900 ml of 0.1 N HCl as the dissolution medium. The set temperature was 37° C. 3 ml samples were taken at different intervals and replaced by fresh medium. Samples were analyzed at 272 nm.

Results:

| | 75% Ranolazine with | 75% Ranolazine |
|---|---|---|
| | pH Dependent | w/out pH dependent |
| Time | Binders | Binders |
| (hr) | Drug Release (%) | Drug Release (%) |
| 0.5 | 17.2 | 9.082 |
| 1 | 25.4 | 14.385 |
| 2 | 36.0 | 23.326 |
| 4 | 49.0 | 35.840 |
| 6 | 57.0 | 46.251 |
| 8 | 63.0 | 54.723 |
| 12 | 73.0 | 69.757 |
| 20 | 87.0 | 88.860 |
| 24 | 93.0 | 93.546 |
| F1 (<15) | | 13.9 |
| F2 (>50) | | 52.3 |

Conclusion

The above table compares the dissolution profile of a sustained release 75% SR ranolazine that has no pH dependent binder present with the 75% SR ranolazine that includes a pH dependent binder, which is the formulation used in clinical testing of ranolazine. The F values are known as “fit factors”, as disclosed in Moore, J. W, and H. H. Flanner, 1996, “Mathematical Comparison of Dissolution Profiles”, Pharmaceutical Technology, 20 (6):64-74, the complete disclosure of which is hereby incorporated by reference. For the formulations to be considered comparable with respect to their dissolution profile, F1 should have a numerical value of less than 15 and F2 should have a numerical value of greater than 50. As can be seen from the above table, the two formulations are comparable using these criteria.

### EXAMPLE 3

Preparation of a Ranolazine SR Formulation with a pH Independent Polymer

Procedure

Ranolazine (750 g), Avicel® (75 g) and HPMC E10M Premium CR (155 g) were mixed together in a granulator (KG-5 high shear mixer) for five minutes at 250 rpm impeller speed and 2500 rpm chopper speed.

260 ml of water was added to the powder mixture at a rate of 100 ml/min, impeller speed of 250 and chopper speed of 2500 rpm.

Powder was massed at impeller speed of 250 rpm and chopper speed 5000 rpm for 5 minutes in order to facilitate agglomeration.

The prepared granules were dried in fluid bed dryer for 25 minutes at an inlet air temperature of 60° C. and nominal air flow setting of 8.

The dried granules were passed through a screen mill using an appropriate screen (0.083 inch screen).

The granules obtained were taken out, weighed and mixed with 2% magnesium stearate (20 g, presifted with 40 mesh) for 3 minutes using a blender (for example, a V-blender).

Granules weighing 666.7 mg were compressed on a Carver Press, then on Stokes 16-Station press with 4 punch set.

Similarly, other Ranolazine SR Formulations are prepared with a pH Independent Polymer, varying the amounts of ranolazine, the pH independent binder, and Avicel.

Dissolution Test

Tablets were tested using USP dissolution apparatus II, stirring at 50 rpm. Using 900 ml of 0.1 N HCl as the dissolution medium. The set temperature was 37° C. 3 ml samples were taken at different intervals and replaced by fresh medium. Samples were analyzed at 272 nm.

Result

| | 75% Ranolazine with pH | 75% Ranolazine w/out pH |
|---|---|---|
| | Dependent Binders | Dependent Binder |
| Time (hr) | Drug Release (%) | Drug Release (%) |
| 0.5 | 17.2 | 11.6 |
| 1 | 25.4 | 16.8 |
| 2 | 36.0 | 25.2 |
| 4 | 49.0 | 36.2 |
| 6 | 57.0 | 48.0 |
| 8 | 63.0 | 55.0 |
| 12 | 73.0 | 67.0 |
| 20 | 87.0 | 83.9 |
| 24 | 93.0 | 89.6 |
| F1 (<15) | | 13.4 |
| F2 (>50) | | 54.5 |

Conclusion

The above table compares the dissolution profile of a sustained release 75% SR ranolazine that has no pH dependent binder (but with Avicel® present) with the 75% SR ranolazine that includes a pH dependent binder, which is the formulation used in clinical testing of ranolazine. The F values are known as “fit factors”, as disclosed in Moore, J. W, and H. H. Flanner, 1996, “Mathematical Comparison of Dissolution Profiles”, Pharmaceutical Technology, 20 (6):64-74, the complete disclosure of which is hereby incorporated by reference. For the formulations to be considered comparable with respect to their dissolution profile, F1 should have a numerical value of less than 15 and F2 should have a numerical value of greater than 50. As can be seen from the above table, the two formulation are comparable using these criteria.

### EXAMPLE 4

Preparation of a Ranolazine SR Formulation with a pH Independent Polymer

Procedure

Ranolazine (750 g), Avicel® (30 g) and HPMC E10M Premium CR (155 g) were mixed together in a granulator (KG-5 high shear mixer) for five minutes at 250 rpm impeller speed and 2500 rpm chopper speed.

260 ml of water was added to the powder mixture at a rate of 100 ml/min, impeller speed of 250 and chopper speed of 2500 rpm.

Powder was massed at impeller speed of 250 rpm and chopper speed 5000 rpm for 5 minutes in order to facilitate agglomeration.

The prepared granules were dried in fluid bed dryer for 25 minutes at an inlet air temperature of 60° C. and nominal air flow setting of 8.

The dried granules were passed through a screen mill using an appropriate screen (0.083 inch screen).

The granules obtained were taken out, weighed and mixed with 2% magnesium stearate (20 g, presifted with 40 mesh) for 3 minutes using a blender (for example, a V-blender).

Granules weighing 666.7 mg were compressed on a Carver Press, then on Stokes 16-Station press with 4 punch set.

Similarly, other Ranolazine SR Formulations are prepared with a pH Independent Polymer, varying the amounts of ranolazine, the pH independent binder, and Avicel.

Dissolution Test

Tablets were tested using USP dissolution apparatus II, stirring at 50 rpm. Using 900 ml of 0.1 N HCl as the dissolution medium. The set temperature was 37° C. 3 ml samples were taken at different intervals and replaced by fresh medium. Samples were analyzed at 272 nm. Result

| | 75% Ranolazine with pH | 75% Ranolazine w/out pH |
|---|---|---|
| | Dependent Binders | Dependent Binder |
| Time (hr) | Drug Release (%) | Drug Release (%) |
| 0.5 | 17.2 | 17.7 |
| 1 | 25.4 | 25.4 |
| 2 | 36.0 | 36.3 |
| 4 | 49.0 | 50.7 |
| 6 | 57.0 | 63.6 |
| 8 | 63.0 | 70.2 |
| 12 | 73.0 | 83.0 |
| 20 | 87.0 | 94.8 |
| 24 | 93.0 | 97.0 |
| F1 (<15) | | 7.60 |
| F2 (>50) | | 62.51 |

Conclusion

The above table compares the dissolution profile of a sustained release 75% SR ranolazine that has no pH dependent binder (but with Avicel present) with the standard 75% SR ranolazine that includes a pH dependent binder, and is the standard formulation used in clinical testing of ranolazine. The F values are known as “fit factors”, as disclosed in Moore, J. W, and H. H. Flanner, 1996, “Mathematical Comparison of Dissolution Profiles”, Pharmaceutical Technology, 20 (6):64-74, the complete disclosure of which is hereby incorporated by reference. For the formulations to be considered comparable with respect to their dissolution profile, F1 should have a numerical value of less than 15 and F2 should have a numerical value of greater than 50. As can be seen from the above table, the two formulations are comparable using these criteria.

### EXAMPLE 5

Preparation of a Ranolazine SR Formulation using a Granulation by Extrusion Method of

Preparation

Procedure

Ranolazine (750 g), Avicel® (105.6), Eudragit® L100-55 (100 g), and HPMC (20 g) were mixed together in KG-5 high shear mixer for five minutes at 250 rpm impeller speed and 2500 rpm chopper speed.

Sodium hydroxide (4.4 g) was dissolved in 230 ml of water and added to the powder mixture at a rate of 100 ml/min, impeller speed of 250 and chopper speed of 2500 rpm.

The remaining amount of water (290 ml) was added at the same rate of sodium hydroxide solution (100 ml/min) and the same speed of impeller (250 rpm) and chopper (2500 rpm).

Powder was massed at impeller speed of 250 rpm and chopper speed of 5000 rpm for five min.

The mass was then transferred to Kenwood Major Classic KM800 extrusion and chopped into small pieces (spheronization).

The prepared granules were dried in fluid bed dryer for 25 minutes at an inlet air temperature of 60° C. and nominal air flow setting of 8.

The dried granules were milled in CoMill with an 0.083 inch screen.

The granules obtained were taken out, weighed and mixed with 2% magnesium stearate (20 g, presifted with 40 mesh) for 3 minutes using V-blender.

Granules weighing 666.7 mg were compressed on a Stokes 16-Station press with 3 punch set.

What is claimed is:

1. A sustained release pharmaceutical formulation comprising:
about 35-49% ranolazine;
a pH dependent binder;
a pH independent binder;
and one or more pharmaceutically acceptable excipients.

2. The formulation of claim 1, wherein the pH dependent binder is methacrylic acid copolymer partially neutralized with a base.

3. The formulation of claim 1, wherein the pH independent binder is hydroxypropyl methylcellulose.

4. The formulation of claim 1, wherein the pharmaceutically acceptable excipients are magnesium stearate and microcrystalline cellulose.

5. The formulation of claim 1, wherein the base is sodium hydroxide, potassium hydroxide, or ammonium hydroxide, in a quantity sufficient to neutralize the methacrylic acid copolymer to an extent of about 1-10%, and ranolazine is present in an amount of about 35-45%.

6. The formulation of claim 1 as a single tablet, comprising:
ranolazine 450-550 mg;
methacrylic acid copolymer 400-450 mg;
hydroxypropyl methylcellulose 10-15 mg
microcrystalline cellulose 240-300 mg;
sodium hydroxide 2-5 mg; and
magnesium stearate 20-30 mg.

7. The formulation of claim 6 as a single tablet, comprising:
ranolazine 500 mg;
methacrylic acid copolymer 437.5 mg;
hydroxypropyl methylcellulose 13.38 mg
microcrystalline cellulose 271.5 mg;
sodium hydroxide 2.63 mg; and
magnesium stearate 25 mg.

8. A sustained release pharmaceutical formulation comprising:
about 35-80% ranolazine;
a pH independent binder;
and one or more pharmaceutically acceptable excipients.

9. The formulation of claim 8, wherein the pH independent binder is hydroxypropyl methylcellulose.

10. The formulation of claim 9, wherein the pharmaceutically acceptable excipient is magnesium stearate.

11. The formulation of claim. 9, wherein the pharmaceutically acceptable excipients are magnesium stearate and microcrystalline cellulose.

12. The formulation of claim 11, comprising:
ranolazine 450-550 mg;
hydroxypropyl methylcellulose 138-170 mg; and
magnesium stearate 12-15 mg.

13. The formulation of claim 12, comprising:
ranolazine 500 mg;
hydroxypropyl methylcellulose 153.3 mg; and
magnesium stearate 13.33 mg.

14. The sustained release pharmaceutical formulation of claim 8, wherein the pH independent binder has a viscosity of about 4,000-12,000 cPs.

15. The formulation of claim 14, wherein the pH independent binder is hydroxypropyl methylcellulose.

16. The formulation of claim 15, wherein the pharmaceutically acceptable excipient is magnesium stearate.

17. The formulation of claim 16, wherein the pharmaceutically acceptable excipients are magnesium stearate and microcrystalline cellulose.

18. The formulation of claim 17, wherein the hydroxypropyl methylcellulose is chosen from Methocel El OM Premium CR grade HPMC or Methocel E4M Premium HPMC.

19. The formulation of claim 18, comprising:
ranolazine 450-550 mg;
hydroxypropyl methylcellulose 138-170 mg; and
magnesium stearate 12-15 mg.

20. The formulation of claim 19, comprising:
ranolazine 500 mg;
hydroxypropyl methylcellulose 153.3 mg; and
magnesium stearate 13.33 mg.

21. A method of treating a cardiovascular disease, comprising administering a sustained release pharmaceutical formulation of claim I in a therapeutically effective amount.

22. The method of claim 21, wherein the cardiovascular disease is chosen from heart failure, including congestive heart failure, acute heart failure, and myocardial infarction, arrhythmias, angina, including exercise-induced angina, variant angina, stable angina, unstable angina, and acute coronary syndrome, diabetes, and intermittent claudication.

23. A method of treating a cardiovascular disease, comprising administering a sustained release pharmaceutical formulation of claim 8 in a therapeutically effective amount.

24. The method of claim 23, wherein the cardiovascular disease is chosen from heart failure, including congestive heart failure, acute heart failure, and myocardial infarction, arrhythmias, angina, including exercise-induced angina, variant angina, stable angina, unstable angina, and acute coronary syndrome, diabetes, and intermittent claudication.
